(19) United States
(12) Patent Application Publication
(10) Pub. No.: US 2011/0065139 A1
(43) Pub. Date: Mar. 17, 2011

(54) DIAGNOSTIC DEVICE FOR IDENTIFYING RUPTURE OF MEMBRANE DURING PREGNANCY

(76) Inventors: Jacob Mullerad, Galil-Ma'aravi (IL); Hagit Frenkel-Mullerad, Galil-Ma'aravi (IL); Ronen Nahary, Galil-Ma'aravi (IL)

(21) Appl. No.: 12/738,714

(22) PCT Filed: Oct. 22, 2008
(86) PCT No.: PCT/IL08/01371

§ 371 Date: Jul. 7, 2010

Related U.S. Application Data
(60) Provisional application No. 60/990,327, filed on Nov. 27, 2007.

(51) Int. Cl. C12Q 1/42; G01N 33/68

(52) U.S. Cl. 435/21; 436/86

(57) ABSTRACT

A diagnostic device is provided for distinguishing between amniotic fluid and urine in female secretion. The device can be employed as a panty shield or can be adhered onto a panty shield or can be employed in a simple pad that is pressed against a substrate provided with female secretion.

### FIELD OF THE INVENTION

The present invention relates to diagnostic methods and devices. More particularly, the present invention relates to diagnostic test and devices for identification of membrane rupture during pregnancy.

### BACKGROUND OF THE INVENTION

Labor is different for every woman, and it can be difficult to pinpoint the moment when it begins. Rather than a single event, it is an entire process, with several physiological changes and events occurring in the body that combine to eventually deliver a baby. One of those changes or events is the rupture of the membrane surrounding the baby in the uterus; which is usually a clear indication that labor is imminent. The rupture of the membrane happens sometimes abruptly and obviously, with a copious flow of amniotic fluid. But often, a pregnant woman may have a ruptured membrane and be very uncertain or unaware whether the membrane has been really ruptured.

There are two main reasons for this. First, the baby's head may act as a cork at the opening of the uterus and so, instead of gushing out, the amniotic fluid will only be slowly released. Moreover, amniotic fluid may leak out drop by drop from a tiny opening of the uterus, and the pregnant woman may not feel the first contractions until hours later. Secondly, in late pregnancy, women often have difficulty controlling their urination. Because of this, a gradual flow of amniotic fluid from the vagina will not be noticed when the woman is accustomed to having small amounts of urine escape involuntarily from time to time.

Wrong and untimely diagnosis of amniotic fluid leakage, especially in high-risk pregnancies, can result in failure to implement proper treatment, which increases the risk to the pregnant women and her fetus. Risks of neonatal consequences of unnoticed amniotic fluid leakage include fetal distress, infection and preterm delivery and can lead to very dangerous sequels for both the mother and fetus.

Currently, the only available way for a pregnant woman to detect at home amniotic fluid is achieved by using an expensive panty liner self-testing diagnostic kit. Thus, almost all pregnant women choose to visit their physicians or the emergency room in a hospital. At the hospital, the tests available today for the identification of amniotic fluid are invasive, indecisive, or expensive and in any case lead to discomfort for the patient. There is a need to greatly reduce the likelihood of membrane rupture being unnoticed at home by using an accurate, fast and inexpensive device that can act also as a tool for the physician to manage the test at the clinic/hospital.

### PRIOR ART

Among several conventional methods available today by physicians, a few examples are described herein. The most widespread method for urine composition analysis is by using a technique known as a “dip-and-read test strip” or “dipstick”. A dipstick is an assay strip that is made up of chemical reagents bonded to a reagent carrier matrix (pad) on a strip. Usually, the strip itself is formed from polymeric materials such as polyethylene, polycarbonate or polystyrene. Each carrier matrix has a different reagent in it. This dipstick is dipped into a urine sample and removed. Upon contact between the reagents imbedded in the matrices and the urine sample, color reactions occur. A dipstick can be designed either as a single pad test strip (for the assay of one analyte) or as a multiple pad test strip (for the assay of several different analytes all together). Dipstick can be used manually or with the appropriate chemistry analyzer. Multiple profile reagent strip for simultaneously or sequentially performing multiple analyses of analyte is disclosed in U.S. Pat. Nos. 4,595,439, 4,526,753, 4,160,008 3,123,443, 3,212,855, 3,814,668, 4,038,485, 3,531,254.

Several such test devices are available in the market. The following is a partial list of dipstick trademarks CLINISTIX, MULTISTIX, KETOSTIX, N-MULTISTIX, DIASTIX, DEXTROSTIX, AUTION STICKS, CHEMSTRIP.

The reagent carrier matrix is usually an absorbent material which allows the liquid sample to move through the matrix. This movement of liquid sample is in response to capillary forces formed in the matrix. During the movement of the liquid through the matrix, it contacts the chemical reagent composition impregnate in the matrix. Thereafter, detectable and measurable color transition occurs. If the dipstick can measure several analytes simultaneously, the color change in each reagent carrier matrix can be correlated to the amount of different analyte in the liquid sample. Manual analysis of the results requires comparison of the color development of the test on the dipstick to a color chart.

The reagent carrier matrix material can be of any substance that can incorporate the chemical reagents necessary to carry out the assay of interest. The preferable matrix should be inert with respect to the chemical reagents and should not alter the sample or the test results. Reagent carrier matrices can be made of many materials, some of these materials are: fiber-containing papers such as filter papers, woven and nonwoven fabrics, synthetic or modified natural polymers, sponge materials, cellulose, glass fiber, microporous membranes, and wood. The reagent matrix can also differ in regards to roughness and smoothness together with softness and hardness. The following list of patents describe the use of different matrices: U.S. Pat. Nos. 3,846,247, 3,552,928, 3,802,842, 3,418,083.

Sewell D L, et al. discusses, among other things, the cost of using the dipstick as a screening method for urinalysis in a scientific paper published in the American Journal of Clinical Pathology Vol. 83 (6) pages 740-743, 1985. The authors state that using a dipstick procedure “cost approximately $0.76 for reagents”. Various devices are described in the literature for the determination of particular urinary analytes with the use of reagent carrier matrices (filter paper, microcapsules, dipstick, etc.). The following list of assay devices utilizing prior art includes dry tablets, dipsticks, or other techniques for the analysis urinary constituents. U.S. Pat. No. 4,147,514 describe the detection of ketone bodies; U.S. Pat. No. 3,146,070 discloses chemical compositions in dry form on a carrier (dipstick) impregnated with a pH indicator for the determination of pH. Methods, composition, and test device for determining the ionic strength or specific gravity of a test sample such as urine are disclosed in the following U.S. Pat. Nos. 4,318,709, 5,403,744.

Jaffe method is a widely known method for the determination of creatinine. This method involves formation of orange-red color with an alkaline picrate solution. U.S. Pat. No. 6,001,656 discloses a device for the assay of creatinine in fluid test samples. The improvement in this patent involves the inclusion of one or more selected quinolines in the reagent formulation. Another method for creatinine determination is described by Benedict and Behre in the Journal of Biological Chemistry (1936) which involves the reaction of 3,5-dinitrobenzoic acid with creatinine in an alkaline medium. Other methods, composition, and test devices for determining creatinine in a liquid sample such as urine are disclosed in the following U.S. Pat. No. 4,215,197 (using an enzymatic composition), U.S. Pat. Nos. 5,662,867, 5,733,787. Suitable materials for the detection of creatinine include picric acid, 3,5-dinitrobenzoic acid, 3,4-dinitrobenzoic acid, 2,4-dinitrobenzene sulfonic acid, (3,5-dinitrobenz)yl alcohol, (3,5-dinitrobenzo)-nitrile, (3,5-dinitrobenz)amide and N,N-diethyl-(3,5-dinitrobenz)amide.

Different methods for protein determination in fluid have been reported. These methods include the Biuret method, Lowry method, Kjeldahl method, dyestuff combination method, fluorometric method and UV method. Of these methods, the Kingbury-Clark method; reported in J. Lab. Clin. Med., 11, 981 (1926) and the Meulemans method; reported in Clin. Chim. Acta, 5, 757 (1960) and the Coomassie brilliant blue method; reported in Anal. Biochem. 72, 248 (1967) are widely used. The Bradford dye assay for protein determination, U.S. Pat. No. 4,023,933, is also used routinely in almost every biochemical laboratory. Generally, protein interacts with substances, principally with dyes such as coomassie brilliant blue, bromphenol blue (tetrabromophenol blue), and eosine as well as metal ions such as copper (II), lead (II) zinc (II) and silver (I). The addition of protein-containing solution to the reaction between a dye and a metal ion gives a spectral change to a dye-metal ion solution. More protein indicators include those described as well as the merocyanine and nitro or nitroso substituted polyhalogenated phenolsulfonephthaleins disclosed in U.S. Pat. No. 5,279,790. Other protein indicators are Fast Green FCF, Light Green SF, pyrogallol red and pyrocatechol violet, bromochlorophenol blue (3′,3″-dibromo-5′,5″-dichlorophenolsulfonephthalein), basic fuchsin, basic violet, martius yellow, phloxine B, methyl yellow, congo red, methyl orange and ethyl orange (4-(4-diethylaminophenylazo)benzenesulfonic acid). The following list of U.S. patents concern with measurement of protein in solution, such as urine, using reagent systems usable in the dipstick method: U.S. Pat. Nos. 5,424,215, 5,593,895, 6,815,210, 4,960,710, 3,485,587, 5,087,575, 4,023,933.

Various dipsticks used for urine testing contain tests for urobilinogen. CHEMSTRIP of Roch diagnostics, and MULTISTIX of Bayer diagnostics are typical examples of such products which include tests for urobilinogen. The classical urobilinogen test, developed by Paul Ehrlich in 1901, employs paradimethylaminobenzaldehyde as a test for which in strongly acid medium produces a brown-orange-red color with Ehrlich's reagent. More background on urobilinogen, Ehrlich reaction and urobilinogen testing is described in, Tietz, Textbook of Clinical Chemistry, W. B. Saunders Company. Examples of U.S. patents and dipsticks for the determination of urobilinogen based on Ehrlich's reaction and the diazonium coupling reaction are: U.S. Pat. Nos. 3,853,466, 3,630,680, 4,665,038, 4,290,771, 3,989,462, 3,814,586. More U.S. patents concern with measurement of urobilinogen in solution, such as urine, using reagent systems usable in the dipstick method are: U.S. Pat. Nos. 4,158,546 and 3,447,905.

The earliest method of alkaline phosphatase test was introduced by Kay in 1930. Later, a popular assay method for the determination of alkaline phosphatase using p-nitrophenyl phosphate introduced in 1946 by Bessey, Lowry and Brock. This method relays on the fact that after exposure to fluids containing alkaline phosphatase, the colorless p-nitrophenyl phosphate is catalytically hydrolyzed into a yellow colored product p-nitrophenol (and phosphate). Thus, the concentration of the enzyme is determined by following the increased intensity of the yellow color of the reaction's product. Alkaline phosphatase activity is naturally present in raw milk, whereas after pasteurization, the enzyme is denatured. So, alkaline phosphatase activity is used as an indicator for proper milk pasteurization. One such dry test of alkaline phosphatase activity in milk is PHOSPHATESMO MI, manufactured by MACHEREY-NAGEL GmbH & Co.

The presence of hemoglobin in urine is called hemoglobinuria, such a condition can occur as a result of lysis of red blood cells (RBS) in the urinary tract. The term hematuria is used when intact RBS are present in the urine. This condition can occur in bleeding in the renal or genitourinary systems. The most widely used tests for the detection of blood in urine or feces depend on the fact that the heme proteins can act as peroxidases. This reaction requires a hydrogen donor molecule. Typical examples of products that include tests for blood detection are MULTISTIX 10SG, HEMOCCULT II, and AUTION STICKS.

More recently, U.S. Pat. No. 4,357,945, issued on Nov. 9, 1982 to Janco for DEVICE FOR TESTING AND RUPTURING AMNIOTIC MEMBRANE, describes a finger-engaging device provided with a pH indicator. Upon exposure to fluids, the indicator changes color if the amniotic membrane has ruptured. U.S. Pat. No. 5,425,377, issued on Jun. 20, 1995 to Caillouette for PH MEASUREMENT OF BODY FLUID, describes a swabbing structure on a stick, provided with a pH indicator for the measurement of vaginal fluid pH.

Several other methods are provided in the following: U.S. Pat. No. 5,554,504 issued on Sep. 10, 1996 to Rutanen for DIAGNOSTIC METHOD FOR DETECTING THE RUPTURE OF FETAL MEMBRANES, describes the detection of insulin-like growth factor binding protein 1 in a vaginal secretion sample. U.S. Pat. No. 5,281,522, issued on Jan. 25, 1994 to Senyei et al. for REAGENTS AND KITS FOR DETERMINATION OF FETAL FIBRONECTIN IN A VAGINAL SAMPLE, describes kits for detection of rupture of membranes by sampling from the vaginal cavity and exposing it to antibodies such as anti-fetal fibronectin antibody and an anti-fibronectin antibody. U.S. Pat. No. 5,096,830, issued on Mar. 17, 1992 to Senyei et al. for PRETERM LABOR AND MEMBRANE RUPTURE TEST describes a method for determining fetal membrane rupture by removing a sample from the vaginal cavity and contacting it with an insoluble support to which anti-fetal antigen antibody is adhered, and the fetal antigen binding to the support is determined.

Several devices involving panty shields or tampons with pH indicators are known. Following here are examples of some of these patents: U.S. Pat. No. 6,149,590 issued on Nov. 21, 2000 to Smith et al. for SYSTEM FOR IDENTIFYING PREMATURE RUPTURE OF MEMBRANE DURING PREGNANCY describes a pad having an upper outer layer, a lower outer layer, and an intermediate pH-responsive component. U.S. Pat. Nos. 6,921,647 and 6,627,394 issued to Kritzman et al. for SECRETION-MONITORING ARTICLE and DIAGNOSTIC PAD describes pads with pH sensitive indicators for the detection of amniotic fluid leakage. U.S. Pat. No. 5,217,444 issued to Schoenfeld for ABSORBENT TAMPON demonstrates an absorbent material containing a pH indicator material indicating by a color change the acidity or alkalinity of a liquid coming into contact with it.

It should be mentioned that tests that measure only the pH of the vaginal secretion for the differentiation of amniotic fluid from urine are not accurate due to the following: 1) The pH of urine may vary from 4.5 to 8 depending on the kidneys homeostatic activity and water intake, and 2) The pH of amniotic fluid ranges from 6.9 to 7.15 in late pregnancy. This overlapping range of pH can lead to false diagnosis that may cause medical complications.

Immunochromatographic tests which are based on antibodies detecting specific proteins in the amniotic fluid or urine, are not commonly available to the general public mainly because of high price due to the monoclonal/polyclonal antibodies which they contains. Generally, Enzymatic methods are also expensive due to the high cost of enzyme production.

### SUMMARY OF THE PRESENT INVENTION

It is an object of the present invention to provide unique devices and methods for differentiating of amniotic fluid from urine that are based on analyzing more then one analyte using dry chemical reactions without the need of enzymatic or immunology methods.

It is another object of the present invention to provide unique devices and methods for differentiating of amniotic fluid from urine that are simple and are of money and time-saving nature that ultimately help solve the dilemma of whether labor may soon begin or not. A woman or a medical caretaker will be able to observe immediately after the first drop of leaking liquid meets the device, whether it contains amniotic fluid or urine.

It is yet another object of the present invention to provide unique devices and methods for differentiating of amniotic fluid from urine that can be used with pregnant female subjects either human or non-human. Determining the onset of labor in non-human females can be even more unpredictable due to the inability of the animal to discuss its condition.

In addition, another object of the present invention is the point of care practice of rapid chemical analysis of biological fluids such as urine, saliva, sweat, cerebrospinal fluid (CSF), milk or fluids from other sources.

It is therefore provided in accordance with a preferred embodiment of the present invention a dry diagnostic device for distinguishing between amniotic fluid and urine in female secretion, the dry diagnostic device comprising:
a base layer;
at least two inert carrier matrices provided on said base layer;
dry reagents provided on said at least two inert carrier matrices wherein said dry reagents are capable of forming a chemical reaction with substances in the female secretion so as to visually distinguishing between the amniotic fluid and urine wherein said dry reagents in each of said at least two carrier matrices is capable of reacting with different substance of said substances.

Furthermore and in accordance with yet another preferred embodiment of the present invention, said at least two carrier matrices are made of absorbent material selected from a group of materials such as fiber-containing papers, woven and non-woven fabrics, synthetic or modified natural polymers, sponge materials, cellulose, glass fiber, micro-porous membranes, wood, micro porous polymer materials such as styrene based copolymer, latex based, cellulose based or cotton based matrices.

Furthermore and in accordance with yet another preferred embodiment of the present invention, said dry reagents are capable of reacting with substances present in the amniotic fluid or in the urine, wherein the substances have concentration markedly higher in one of the amniotic fluid or urine than their concentration in the former.

Furthermore and in accordance with yet another preferred embodiment of the present invention, said substances include substances such as creatinine, alkaline phosphatase, total protein, urea, urobilinogen and blood.

Furthermore and in accordance with yet another preferred embodiment of the present invention, one of the dry reagents in one of said at least two carrier matrices is capable of reacting with creatinine.

Furthermore and in accordance with yet another preferred embodiment of the present invention, said one of the carrier matrices comprises two reagent layers; one of which contains creatinine sensitive dye fixed with a dye fixing agent and a second one containing a buffer capable of keeping said one of the test zones in a relatively high pH value.

Furthermore and in accordance with yet another preferred embodiment of the present invention, said one of the carrier matrices comprises a unique layer containing a creatinine sensitive dye, buffer, and dye fixing agent.

Furthermore and in accordance with yet another preferred embodiment of the present invention, said creatinine sensitive dye is selected from a group of dinitro derivatives such as 3′5′-dinitrobenzoic acid, 2′4′-dinitrobenzoic acid, 3′5′-dinitrobenzotrifluoride, 3′5′-dinitrobenzamide, 3′5′-dinitrobenzoyl-phenyl glycine, 3,5-dinitrohydroxyphenylpropionic acid.

Furthermore and in accordance with yet another preferred embodiment of the present invention, said dye fixing agent selected from polymerized quaternary ammonium cations (quats) such as polydiallyldimethylammoniumchloride, polymonoallyltrimethylammoniumchloride, polytrimethylaminoethylmethacrylatechloride, polyvinylbenzyltrimethylammoniumchloride, polyvinylmβthylpyridine-chloride.

Furthermore and in accordance with yet another preferred embodiment of the present invention, said creatinine sensitive dye and said dye fixing agent are buffered so as to keep a stable pH in a range of about 9 to 13.5.

Furthermore and in accordance with yet another preferred embodiment of the present invention, a buffer is selected from a group of sodium metasilicate, sodium hydroxide, potassium hydroxide, calcium hydroxide, sodium hydroxide-potassium chloride, potassium carbonate, glycine-sodium hydroxide, and sodium borate.

Furthermore and in accordance with yet another preferred embodiment of the present invention, one of the dry reagents in one of the carrier matrices is capable of reacting with total protein.

Furthermore and in accordance with yet another preferred embodiment of the present invention, said one of the dry reagents is a dye selected from a group of 3′,3″,5′,5″-Tetrabromophenolsulfonephthalein, coomassie brilliant blue, Fast Green, Light Green, Pyrogallolsulfonephthalein (pyrogallol red), Pyrocatecholsulfonphthalein (Pyrocatechol Violet), 3′,3″-Dibromo-5′,5″-dichlorophenolsulfonephthalein, Fuchsin acid, 2,4-Dinitro-1-naphthol (martius yellow) phloxine B, congo red, ethyl orange and methyl orange.

Furthermore and in accordance with yet another preferred embodiment of the present invention, the device further comprising a buffer such as potassium citrate, potassium chloride, potassium sulfate, potassium iodate or potassium phosphate.

Furthermore and in accordance with yet another preferred embodiment of the present invention, the device further comprising metal ion selected from Copper, lead, Zink, Silver.

Furthermore and in accordance with yet another preferred embodiment of the present invention, one of the dry reagents in one of the carrier matrices is capable of reacting with alkaline phosphatase.

Furthermore and in accordance with yet another preferred embodiment of the present invention, said one of the dry reagents Alkaline phosphates' substrate selected from p-nitrophenyl phosphate, indoxyl phosphate, 4-methylumbelliferyl phosphate and alpha-naphthyl-phosphate.

Furthermore and in accordance with yet another preferred embodiment of the present invention, one of said dry reagents is a pH sensitive reagent.

Furthermore and in accordance with yet another preferred embodiment of the present invention, said dry reagents cannot exit the carrier matrices.

Furthermore and in accordance with yet another preferred embodiment of the present invention, said at least two carrier matrices are covered by a protective layer.

Furthermore and in accordance with yet another preferred embodiment of the present invention, said protective layer is transparent.

Furthermore and in accordance with yet another preferred embodiment of the present invention, said protective layer can be made of a thin “one-way structure” membrane permeable to liquids flowing to said at least two carrier matrices and prevents flow of reagents outwardly from the device.

Furthermore and in accordance with yet another preferred embodiment of the present invention, a sticky backing layer is provided beneath said base layer.

Furthermore and in accordance with yet another preferred embodiment of the present invention, said sticky backing layer is adjacently provided with an outer protective layer.

Furthermore and in accordance with yet another preferred embodiment of the present invention, said at least two carrier reagent matrices are organized in substantially parallel lines.

Furthermore and in accordance with yet another preferred embodiment of the present invention, said at least two reagent carrier matrices are organized in concentric lines.

In addition and in accordance with yet another preferred embodiment of the present invention, said at least two reagent carrier matrices are surrounded by adhesive material so as to allow adhering the diagnostic device when said at least two test zones are opposite a vaginal canal of a female animal.

### BRIEF DESCRIPTION OF THE FIGURES

Some embodiments of the invention are herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 illustrates a cross sectional view of the diagnostic device in accordance with a preferred embodiment of the present invention.

FIGS. 2-5 illustrate diagnostic devices for adhering onto women panties in accordance with preferred embodiments of the present invention.

FIGS. 6a-d illustrate diagnostic pads in accordance with preferred embodiments of the present invention.

FIG. 7 illustrates a veterinary diagnostic pad in accordance with another preferred embodiment of the present invention.

### DETAILED DESCRIPTION OF THE INVENTION

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

The terms “comprises”, “comprising”, “includes”, “including”, and “having” together with their conjugates mean “including but not limited to”

The term “consisting of” has the same meaning as “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

The present invention provides unique and novel devices to distinguish amniotic fluids from urine in a simple and fast manner so as to allow a pregnant woman to know her pregnancy condition. According to one aspect of the present invention, a device that resembles an ‘adhesive plaster’, which is a disposable strip containing sensitive chemical indicators, is used during last months of pregnancy. The strip is relatively small in its dimensions and comprises at least two layers; an adhesive backing and an absorbing material containing the indicators. The woman using the device simply adheres it to her panties with the test zones facing up towards the body. In case the woman uses panty liner, she can place the strip on top of it. The rapid chemical reactions in the test zones of the device cause a distinct color change in the case of amniotic fluid leakage and a clearly different color change when urine has contacted the test zones. The device of the present invention acts as a diagnostic tool to allow the customer to detect whether the secretion contains amniotic fluid or only urine, according to a known color index supplied with the device.

According to a second aspect of the present invention, a device resembling a pad containing sensitive indicators is used independently of contacting the woman body. This device can be used by pressing it against wet underwear or a wet panty liner after leakage of fluids has been noticed. This will cause the reagents in the reagent carrier matrices to come in contact with the body fluids and almost immediately after the leaking liquid meets the diagnostic device, chemical reactions occur in the reagent carrier matrices and cause a color change. Again, the woman (or caregiver) using this device will be able to see whether the liquid contains amniotic fluid or only urine, according to a known color index supplied with the device.

In accordance with a third aspect of the present invention, a device that resembles a panty-liner is containing the sensitive indicators that allow the distinction between amniotic fluid and urine. The pregnant woman will attach this product to her underwear and will get on with her day. The indicator reagent areas should be positioned directly opposite the vagina. This way, the sensitive panty-liner will be in a close contact with the woman body fluids.

In all aspects of the invention, if storage is needed following collection, the sample can be transferred to a suitable container for storage. Alternatively, immediate processing of the sample can be performed. If used, the sample is placed directly on the reagent carrier matrices of the device and testing is performed within minutes of sample collection.

In accordance with another aspect of the present invention, a veterinary device is provided. The device containing the sensitive reagents will be directly attached to a non-human female vagina opening. This may be achieved simply by a bandage design in which the adhesive zone is around or on the sides of the reagent carrier matrices test zone. Thus, a close contact with the animal body fluids will be achieved. Using a device without direct contact with the animal body can also be achieved by obtaining a fluid sample with a swab having a fibrous tip or by suction or lavage device, and applying it to the indicators areas on the device.

In accordance with a preferred embodiment of the present invention, the determination whether amniotic fluid or urine is present in the woman's secretion relies upon several non-enzymatic and non-immunological separated reactions that can be determined as associated with either urine or amniotic fluid by their distinguished color. In most of the tests available today for the identification of the cause of wetness during pregnancy, concentration difference of only one analyte in urine or amniotic fluid is measured. In the present invention, the existence of at least two of the following substances: protein, creatinine, urea, urobilinogen, blood, and alkaline phosphatase are identified as well as pH. Basically, it was identified that protein and alkaline phosphatase are present in higher concentrations in amniotic fluid relative to urine while creatinine, urea and urobilinogen are present in higher concentrations in urine relative to amniotic fluid. Blood may exist in amniotic fluid in higher concentration than in normal urine. Chemicals that can be used in order to distinguish between the fluids in accordance with a preferred embodiment of the present invention are ones that upon binding or reacting with one of the substances indicated herein or act upon a certain environment undergo a change in spectroscopic properties.

Therefore, diagnostic device that comprises reagent carrier matrices provided with chemical indicators according to the present invention is capable of detecting at least two of the following substances: protein, creatinine, urobilinogen, urea, blood, and alkaline phosphatase as well as pH value. The method of the present invention is based on the following facts 1) The concentration of total protein in amniotic fluid is normally substantially 15 times higher than its concentration in urine, 2) The concentration of creatinine and urobilinogen in urine is normally about 10 times more than their concentration in amniotic fluid, and 3) The concentration of urea in urine is normally more than 2 times its concentration in amniotic fluid, 4) The concentration of alkaline phosphatase in amniotic fluid is normally more than 7 times higher than the concentration in urine, 5) During rupture of the fetal membranes, the amniotic fluid coming out may contain blood, which is in contrast to urine of healthy woman. Therefore, even if a small amount of amniotic fluid is present in vaginal secretion and comes in contact with a pad of the present invention, the pad allows the diagnostic of fetal membrane rupture with an extremely high accuracy. Other substances with similar concentration differences may also be detected.

The present invention is further illustrated by the following examples of devices provided with sensitive indicators for the detection and distinction of urine or amniotic fluid leakage.

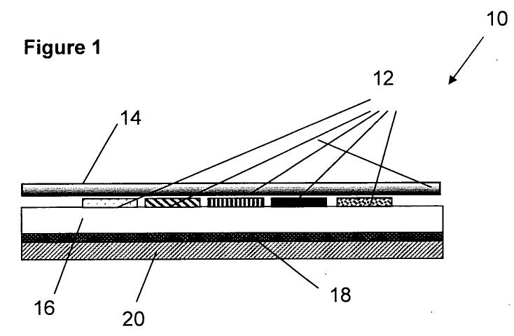

Reference is made to FIG. 1 illustrating a diagnostic device in accordance with a preferred embodiment of the present invention, in an upper view and cross sectional view, respectively. A device 10 has at least two, and preferably several reagent carrier matrices 12. FIG. 1 illustrates a device that is adapted to be attached to the woman's body and therefore covered with a first layer 14 of soft and comfortable material that does not irritate skin upon contact so it can be worn in a woman panty. First layer 14 prevents direct contact between reagents that are provided in within device 10 and the adjacent skin.

It should be noted that first layer 14 may be transparent so as to allow easy visual distinction of the colors formed in layers beneath it.

Device 10 is further comprises with a supportive base layer 16 that can support reagent carrier matrices 12 capable of carrying out the assays of interest. Reagent carrier matrices 12 are preferably an absorbent material that allows the liquid sample to move through the matrix. Reagent carrier matrices 12 should be inert with respect to the chemical reagents and should not alter the sample or the test results.

Optionally, reagent carrier matrices 12 can be made of many materials, for example: fiber-containing papers such as filter papers, woven and nonwoven fabrics, synthetic or modified natural polymers, sponge materials, cellulose, glass fiber, micro-porous membranes, and wood. Additional materials can be micro porous polymer materials such as styrene based copolymer, latex based, cellulose based or cotton based matrices.

The reagent carrier matrices can also be different in characteristics such as roughness, smoothness, softness, and hardness.

It should be noted that in the manufacturing process, the reagent carrier matrices can be made from several layers, some of which carry different reagents in different areas of the test zone. Any combination of the supportive base layer and additional matrices carrying the indicators are covered by the scope of the present invention and by no means limit the scope of the present invention.

Beneath supportive base layer 16, a sticky backing layer 18 is provided and an adjacent outer protective layer 20 is also provided. Those two layers are similar in nature to the layers that are provided in protection panty shields.

Reagent carrier matrices 12 are organized preferably in groups wherein each group is provided with indicators capable of indicating one of the substances that were listed herein before. Following are examples of chemical reagents and methods of preparing the test zone for each:

### Examples

### Creatinine Test Zone Preparation

Creatinine concentration in urine is normally about 10 times higher than its concentration in amniotic fluid. Creatinine dry test is made in two optional ways:
Two reagent layers system that contains fixed creatinine sensitive dye that is placed on one matrix and a buffer that keeps the system in a relatively high pH value is placed on a second matrix.
One reagent layer system that contains a creatinine sensitive dye, buffer, fixing agents all placed in one matrix.

The creatinine sensitive dye can be one of dinitro derivatives such as: 3′5′-dinitrobenzoic acid, 2′4′-dinitrobenzoic acid, 3′5′-dinitrobenzotrifluoride, 3′5′-dinitrobenzamide, 3′5′-dinitrobenzoyl-phenyl glycine, 3,5-dinitrohydroxyphenylpropionic acid.

The sensitive dye fixing agent preferably includes polymerized quaternary ammonium cations (quats) such as: polydiallyldimethylammoniumchloride (Poly DADMAC), polymonoallyltrimethylammoniumchloride, polytrimethylaminoethylmethacrylatechloride, polyvinylbenzyltrimethylammoniumchloride, polyvinylmβthylpyridine-chloride.

The buffer should be a strong base capable of keeping a stable pH in a range of about 9 to 13.5. examples for such buffers are sodium metasilicate, sodium hydroxide, potassium hydroxide, calcium hydroxide, sodium hydroxide-potassium chloride, potassium carbonate, glycine-sodium hydroxide, and sodium borate.

Optionally, non volatile solid reagents are added to the sensitive dye in order to improve the reaction.

### Two Reagent Layers System; Example 1

Creatinine test indicator consisting of two reagent carrier matrices was mounted on the diagnostic device base layer. The first matrix (can be Whatman filter paper or napkin paper) was impregnated with creatinine sensitive dye, 3′5′-dinitrobenzoic acid (dBA), and poly DADMAC (optional) as a fixing agent that were dissolved in water. The dBA stock solution was mixed in sodium carbonate buffer. A second reagent matrix was impregnated with Sodium Metasilicate buffer. After both reagent matrices were dry, the first reagent matrix was laid on the second reagent matrix that was impregnated with Sodium Metasilicate buffer. Both dry reagent matrices were tightened on the base layer of the device.

After a drop of vaginal secretions meet the dry reagent carrier matrix, a distinct color change will identify the liquid and indicate whether it contains amniotic fluid or urine.

### Two Reagent Layers System; Example 2

Two reagent absorbent matrices were impregnated each in one solution, dried and were tightened on a base layer of the device. The first reagent matrix was impregnated with solution 1 that consist of dBA (stock solution was dissolved in Sodium Hydroxide) as a creatinine sensitive dye, non volatile solid reagent and Poly DADMAC (optional) as a fixing agent. The second reagent matrix was impregnated with solution 2 that include Sodium Hydroxide. Both reagent matrices were dried. Dry reagent matrix with solution 1 was placed above dry reagent matrix with solution 2 and both of them were tighten on the base layer of the device.

### One Reagent Layers System; Example 1

As mentioned before, “one reagent layers system” has the same rational as the “two reagent layers system” but contains all the chemicals on one reagent carrier matrix. In both cases, the goal is differentiation between urine and amniotic fluid using creatinine concentration.

Creatinine test device was prepared from the same absorbent and support carriers as in the two reagent layers system. The reagent carrier matrix consists of a creatinine sensitive dye, non volatile solid reagent, a fixing agent (optional) and a buffer that was dried and mounted on the carrier matrix. There was a preferable use of 3′5′-dinitrobenzoic acid (stock solution was made in acetonitrile) as a sensitive dye, Poly DADMAC as a fixing agent (optional) and Potassium Hydroxide (in ethyl alcohol solution) as a buffer. The reagents were dried all together and were ready for urine or amniotic fluid sample test.

### One Reagent Layers System; Example 2

An emulsified solution of 3′5′-dinitrobenzoic acid, Poly DADMAC (optional), Styrene Acrylic acid, Sodium Metasilicate and a non volatile solid reagent was prepared. A thin layer of the emulsion was spread on a latex based matrix, and dried. Dry matrices were tightening on the support base layer polymer of the device.

### Total Protein Test Zone Preparation

The concentration of total protein in amniotic fluid is normally about 15 times more than its concentration in urine. Interaction of proteins with substances, principally dyes and metal ions, causes a spectral change to a dye-metal ion solution. In implementing the device of the present invention, a group of dyes and metal ions such as: 3′,3″,5′,5″-Tetrabromophenolsulfonephthalein, coomassie brilliant blue, Fast Green, Light Green, Pyrogallolsulfonephthalein (pyrogallol red), Pyrocatecholsulfonphthalein (Pyrocatechol Violet), 3′,3″-Dibromo-5′,5″-dichlorophenolsulfonephthalein, Fuchsin acid, 2,4-Dinitro-1-naphthol (martius yellow), Copper, lead, Zink, Silver, phloxine B, congo red, ethyl orange and methyl orange can be used.

The reagent carrier matrix of the total protein test device is an absorbent carrier that can be one of the matrices already mentioned, for example micro porous polymer material such as styrene based copolymer, latex based, cellulose based or cotton based matrices. The reagent carrier matrix can be polymerized urethane-based compound (as described in U.S. Pat. No. 5,124,266) incorporating an indicator reagent compound capable of interacting with proteins to produce a visually detectable response.

In order to reduce the variability due to urine density differences, the test solution may include a low pH potassium salt based buffer such as potassium citrate, potassium chloride, potassium sulfate, potassium iodate or potassium phosphate.

### Total Protein Test; Example

The total protein solution test comprises a combination of two solutions: the dye reagent solution and the buffer. For the dye solution, 3′,3″,5′,5″ Tetrabromophenolsulfonephthalein was used and was dissolved in a weak organic acid such as citric acid. The buffer, potassium citrate, was tittered with the same weak organic acid to a low pH value of around 3.5.

The dye solution was then diluted with the buffer solution in a wide ratio scale. After the dilution, a very thin layer of the resulting solution was spread on a reagent carrier matrix and dried. While a urine and amniotic fluid comes in contact with the reagents imbedded in the device, a distinct color reaction can differentiate between them.

### pH Test Zone Preparation

As mentioned herein before, the preparation is placed on an absorbent reagent carrier matrix of any type. The reagent matrix is impregnated with a pH indicator for the measurement of fluid pH. The urine pH may vary from 4.5 to 8 while the amniotic fluid pH ranges from 6.9 to 7.15 in late pregnancy. This pH range can be check with one indicator or two different pH indicators; a low pH indicator and a middle pH indicator.

According to their useful pH range, for the low pH, Methyl yellow, Methyl orange, Methyl red, Bromofhenol Blue, Tetrabromphenol blue or Bromcresol green can be used. For the middle pH range, Cresol Red, Nitrizine, Bromthymol blue, Neutral red, Rosolic acid, α-Naphtholphthalein or phenol red can be used.

### pH Test; Example

Cresol Red was dissolved in water and was impregnated on 3M paper (blotting paper ra-reeve angel®). After the material is fully impregnated, the matrix is dried.

### Alkaline Phosphatase Test Zone Preparation

Alkaline phosphatase activity in amniotic fluid is much higher then in normal urine. Therefore, this activity can be used to differentiate and identify amniotic fluid from urine in vaginal secretion by using a dry test by which direct contact with such secretions is resulted by a distinct and different color formation for the secretions. When drop of vaginal secretion meets the dry matrix reaction zone, a distinct color change will identify the secretion content and indicate whether it contains amniotic fluid or urine.

### Alkaline Phosphatase Test; Example

The composition of the reaction reagent matrix for the detection of Alkaline phosphatase contained dry buffered solution of Alkaline phosphates' substrate such as p-nitrophenyl phosphate, indoxyl phosphate, 4-methylumbelliferyl phosphate and alpha-naphthyl-phosphate and may also contain sensitive indicators such as bromocresol green.

Reference is now made to FIGS. 2-5 illustrating diagnostic device for adhering onto women panties in accordance with preferred embodiments of the present invention. All diagnostic devices shown in FIGS. 2-5 comprises a base layer 22 that includes layers similar to the layers that are shown in FIG. 1 that allows the device to be used in the panty of a women, adhered directly to the woman's panty or adhered onto a panty shield while the layer with the test zone, which will be explained herein after, is directly positioned beneath the vaginal canal of the woman. In order to view the device, the protective layer is removed from the drawings. FIG. 2 illustrates a diagnostic device 20 having test zones that are divided into five reagent carrier matrices 24-32 wherein each of the reagent carrier matrices is provided with diagnostic indicator that is capable of identifying a specific substance as elaborated herein before—e.g. creatinine test, protein test, alkaline phospatase test ed.

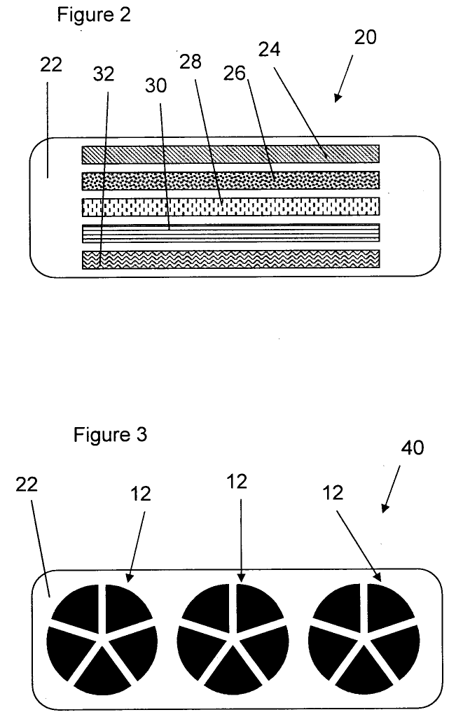

FIG. 3 illustrates a diagnostic device 40 having three distinct test zones 12. Each zone is divided into five different reagent carrier matrices wherein each one is provided with different indicator capable of distinguishing between urine and amniotic fluid.

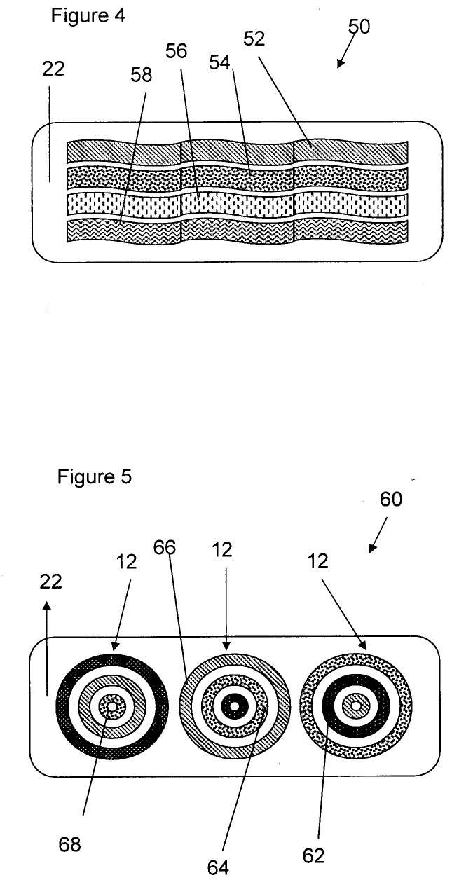

FIG. 4 illustrates a diagnostic device 50 similar to device 20 wherein different indicators are in 4 reagent carrier matrices 54-58.

FIG. 5 illustrates a diagnostic device 60 having test zones 12, each having a reagent carrier matrices 62-68 provided with different indicators so as to distinguish between urine and amniotic fluid.

As mentioned herein before, according to the present invention, the device may contain two or more reagent carrier matrices in lines, circles or any other formation so at least two indicators are changed in their color so as to be able to clearly distinguish between the urine and the amniotic fluid.

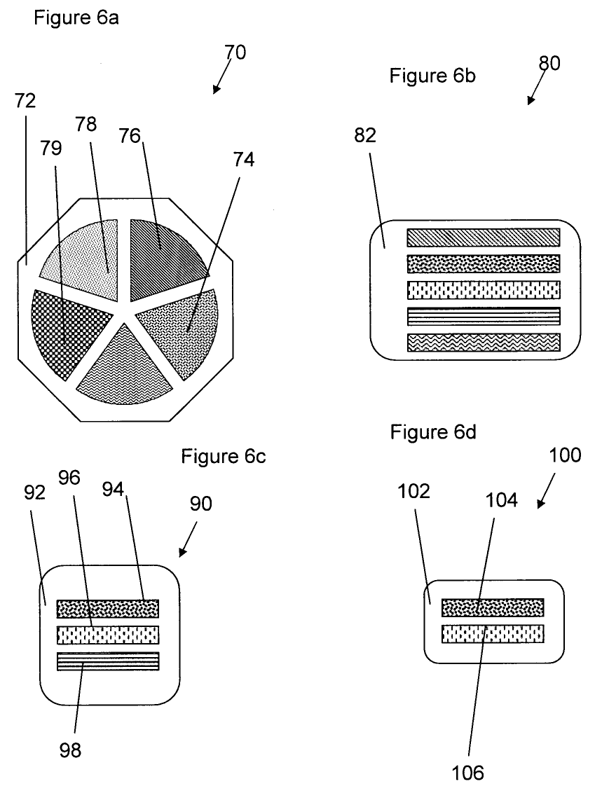

Reference is now made to FIGS. 6a-d illustrating diagnostic pads in accordance with preferred embodiments of the present invention. As mentioned herein before, a pad can be used in a way that it is pressed against a regular wet panty shield or wet panties, as an example, so as to prevent contact between the skin and the pad. Basically, the pad is built similarly to the adhered one, however, there is no adherence layer on the pad.

FIG. 6a illustrates a pad 70 having a base layer 72 onto which different reagent carrier matrices 74-79 provided with different indicators. Each reagent carrier matrices is capable of identifying a certain substance as explained herein before.

FIG. 6b illustrates a pad with base layer 82 onto which reagent carrier matrices 94-98 or 104-106 are arranged in different arrangement. FIGS. 6c and 6d illustrate additional embodiments of pads 90 and 100 provided with less reagent carrier matrices zones, respectively.

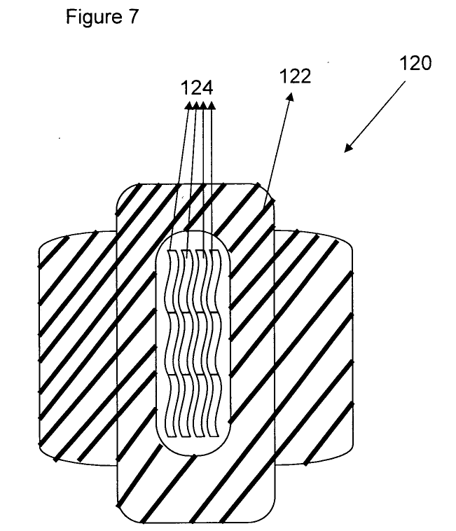

Reference is now made to FIG. 7 illustrating a veterinary diagnostic pad in accordance with another preferred embodiment of the present invention. Veterinary device 120 is basically similar to the diagnostic device used for humans however, the figure shows an upper view of pad 120 in which the adhesive zone 122 is around the test zones 124.

1-27. (canceled)

28. A dry diagnostic device for distinguishing between amniotic fluid and urine in female secretion, the dry diagnostic device comprising: a base layer: two inert carrier matrices provided on said base layer; dry reagents provided on said two inert carrier matrices wherein said dry reagents are capable of forming a chemical reaction with substances in the female secretion selected from two or more of a group comprising creatinine, alkaline phosphatase and total protein, so as to visually distinguish between the amniotic fluid and urine, with the proviso that said reagents are neither enzymes nor antibodies.

29. The dry diagnostic device as claimed in claim 28, wherein said two carrier matrices each independently comprise absorbent material selected from a group of materials comprising fiber-containing papers, woven and nonwoven fabrics, synthetic or modified natural polymers, sponge materials, cellulose, glass fiber, micro-porous membranes, wood, micro porous polymer materials comprising styrene based copolymer, latex based, cellulose based or cotton based matrices.

30. The dry diagnostic device as claimed in claim 28, wherein said dry reagents are capable of reacting with substances present in the amniotic fluid or in the urine, wherein the substances independently have concentration markedly higher in amniotic fluid than in urine or markedly higher in urine than in amniotic fluid.

31. The dry diagnostic device as claimed in claim 28, wherein the carrier matrix on which reagent capable of reacting with creatinine is provided comprises two reagent layers; one of which contains creatinine sensitive dye fixed with a dye fixing agent and a second one containing a buffer capable of keeping said one of the test zones in a relatively high pH value.

32. The dry diagnostic device as claimed in claim 30, wherein said one of the carrier matrices comprises a layer comprising a creatinine sensitive dye, buffer, and dye fixing agent.

33. The dry diagnostic device as claimed in claim 31 wherein said creatinine sensitive dye is selected from a group of dinitro derivatives comprising 3′5′-dinitrobenzoic acid, 2′4′-dinitrobenzoic acid, 3′5′-dinitrobenzotrifluoride, 3′5′-dinitrobenzamide, 3′5′-dinitrobenzoyl-phenyl glycine, 3,5-dinitrohydroxyphenylpropionic acid.

34. The dry diagnostic device as claimed in claim 32 wherein said creatinine sensitive dye is selected from a group of dinitro derivatives comprising 3′5′-dinitrobenzoic acid, 2′4′-dinitrobenzoic acid, 3′5′-dinitrobenzotrifluoride, 3′5′-dinitrobenzamide, 3′5′-dinitrobenzoyl-phenyl glycine, 3,5-dinitrohydroxyphenylpropionic acid.

35. The dry diagnostic device as claimed in claim 31, wherein said dye fixing agent is a polymerized quaternary ammonium cations (quats) selected from a group comprising: polydiallyldimethylammonium chloride, polymonoallyltrimethylammonium chloride, polytrimethylaminoethylmethacrylate chloride, polyvinylbenzyltrimethylammonium chloride, polyvinylmethylpyridine-chloride.

36. The dry diagnostic device as claimed in claim 32, wherein said dye fixing agent is a polymerized quaternary ammonium cations (quats) selected from a group comprising: polydiallyldimethylammonium chloride, polymonoallyltrimethylammonium chloride, polytrimethylaminoethylmethacrylate chloride, polyvinylbenzyltrimethylammonium chloride, polyvinylmethylpyridine-chloride.

37. The dry diagnostic device as claimed in claim 31, wherein said creatinine sensitive dye and said dye fixing agent are buffered so as to keep a stable pH in a range of about 9 to 13.5.

38. The dry diagnostic device as claimed in claim 32, wherein said creatinine sensitive dye and said dye fixing agent are buffered so as to keep a stable pH in a range of about 9 to 13.5.

39. The dry diagnostic device as claimed in claim 31, wherein the buffer is selected from a group comprising: sodium metasilicate, sodium hydroxide, potassium hydroxide, calcium hydroxide, sodium hydroxide-potassium chloride, potassium carbonate, glycine-sodium hydroxide, and sodium borate.

40. The dry diagnostic device as claimed in claim 32, wherein the buffer is selected from a group of sodium metasilicate, sodium hydroxide, potassium hydroxide, calcium hydroxide, sodium hydroxide-potassium chloride, potassium carbonate, glycine-sodium hydroxide, and sodium borate.

41. The dry diagnostic device as claimed in claim 28, wherein one of the dry reagents in one of the carrier matrices is capable of reacting with total protein.

42. The dry diagnostic device as claimed in claim 41, wherein said one of the dry reagents is a dye selected from a group of 3′,3″,5′,5″-Tetrabromophenolsulfonephthalein, coomassie brilliant blue, Fast Green, Light Green, Pyrogallolsulfonephthalein (pyrogallol red), Pyrocatecholsulfonphthalein (Pyrocatechol Violet), 3′,3″-Dibromo-5′,5″-dichlorophenolsulfonephthalein, Fuchsin acid, 2,4-Dinitro-1-naphthol (martius yellow) phloxine B, congo red, ethyl orange and methyl orange.

43. The dry reagent device as claimed in claim 42, further comprising a buffer selected from a group comprising potassium citrate, potassium chloride, potassium sulfate, potassium iodate or potassium phosphate.

44. The dry diagnostic device as claimed in claim 28, wherein one of the dry reagents in one of the carrier matrices is capable of reacting with alkaline phosphatase.

45. The dry diagnostic device as claimed in claim 44, wherein said one of the dry reagents is selected from p-nitrophenyl phosphate, indoxyl phosphate, 4-methylumbelliferyl phosphate and alpha-naphthyl-phosphate.

46. The dry diagnostic device as claimed in claim 28, wherein said at least two carrier matrices are covered by a transparent protective layer.

47. The dry diagnostic device as claims in claim 28, wherein a sticky backing layer is provided beneath said base layer.

48. The dry diagnostic device as claimed in claim 47, wherein said sticky backing layer is adjacently provided with an outer protective layer.

49. The dry diagnostic device as claimed in claim 42, further comprising metal ion selected from a group comprising: Copper, lead, Zinc, Silver.

50. A kit comprising: the device of claim 28 and a color index configured to allow distinguishing between the amniotic fluid and urine by comparing the colors of the device after contact of the device with amniotic fluid or urine to the colors of the index.
